(19) United States
(12) Patent Application Publication
(10) Pub. No.: US 2026/0301955 A1
(43) Pub. Date: Oct. 1, 2026

(54) COMPUTER-IMPLEMENTED METHOD, DIGITAL HEALTH PLATFORM, ARTIFICIAL INTELLIGENCE POWERED CLINICAL DECISION SUPPORT SOFTWARE TOOL AND NON-TRANSITORY COMPUTER-READABLE MEDIUM

(71) Applicant: Siemens Healthineers AG, Forchheim (DE)

(72) Inventors: Bernd FRANKE, Gremsdorf (DE); Lutz DOMINICK, Eggolsheim (DE)

(73) Assignee: Siemens Healthineers AG, Forchheim (DE)

(21) Appl. No.: 19/577,454

(22) Filed: Mar. 25, 2026

(30) Foreign Application Priority Data
Mar. 26, 2025 (DE) 10 2025 111 733.0

(51) Int. Cl. G16H 50/20; G16H 10/60

(52) U.S. Cl. CPC G16H 50/20; G16H 10/60

(57) ABSTRACT

A computer-implemented method and a digital health platform for processing clinical data by an artificial intelligence algorithm grid generated by a relevance grid builder, the method comprising executing the artificial intelligence algorithm grid for a workflow step of a predefined clinical workflow of a customer to produce an intermediate clinical result having a clinical relevance evaluated by a relevance detector; and determining iteratively another artificial intelligence algorithm grid to be executed for a next workflow step of the clinical workflow until configurable end conditions are met.

### CROSS-REFERENCE TO RELATED APPLICATION(S)

The present application claims priority under 35 U.S.C. § 119 to Germany Patent Application No. 10 2025 111 733.0, filed Mar. 26, 2025, the entire contents of which is incorporated herein by reference.

### FIELD

One or more example embodiments relates to a digital health platform adapted to run a computer-implemented method for processing clinical data by an artificial intelligence algorithm grid. One or more example embodiments relates to a digital health platform adapted to run a computer-implemented method for processing clinical data by artificial intelligence algorithms. One or more example embodiments relates to a digital health platform, an artificial intelligence powered clinical decision support software tool and a computer-program product.

### TECHNICAL RELATED ART

Conventional tools are designed to assist radiologists and healthcare professionals in analyzing medical images more efficiently and accurately using artificial intelligence and machine learning algorithms. They help in tasks such as image segmentation, anomaly detection, and clinical decision support, thereby aiming to improve diagnostic accuracy and workflow in radiology departments. These tools are part of the broader trend towards integrating artificial intelligence into medical imaging to enhance patient care.

Currently artificial intelligence applications in the medical domain are scattered across multiple products and are available in different deployments, such as cloud, edge, and on-premises exclusively. Therefore different kinds of artificial intelligence processing routines are deployed in multiple products and hence different clinical results are created, which is not acceptable in a customer workflow. There is also a trend to smarter, more focused, faster artificial intelligence algorithms with smaller resource footprints. Artificial intelligence comprises a wide variety of techniques and technologies, which creates a contradiction to solutions that do not take the fast-evolving structure of the AI landscape into account. National guidelines have partitioned organs and body regions into standardized sections for clinical purposes.

The automation of artificial intelligence resolves these issues by providing a technical solution with an artificial intelligence engine running for all types of deployments of artificial intelligence algorithms including deployments in a cloud, on edge devices and on-premises. Providing the same clinical results in all deployment environments, requires the artificial intelligence algorithms to run in any deployment scenario and to orchestrate efficiently the execution of the deployed artificial intelligence algorithms.

Conventional solutions are setup and configured manually to enable product specific embodiments of AI-usage, attached and specific to the available product deployments and the current architecture of each product. To mitigate update issues, artificial intelligence algorithms are updated manually in the various products but with different timelines. A scanner typically provides an update every two year. Products require a complete product update and do not allow updating specific AI processing only, from a regulatory perspective.

### SUMMARY

Against this background, a problem addressed by example embodiments is that of providing a computer-implemented method for processing clinical data efficiently and being in particular available over the complete workflow of a customer in all deployment scenarios.

### BRIEF DESCRIPTION OF THE DRAWINGS

The present invention is described in greater detail in the following on the basis of the embodiments shown in the schematic figures of the drawings, in which:

FIG. 1 shows a block diagram of a digital health platform according to one or more example embodiments of the present invention;

FIG. 2 shows schematically a technical context of the AI processing performed by the method according to one or more example embodiments of the present invention;

FIG. 3 shows an integration layer of an artificial intelligence engine adapted to the method according to one or more example embodiments of the present invention;

FIG. 4 shows the pipeline approach of the method according to one or more example embodiments of the present invention for processing clinical data.

The appended drawings are intended to provide further understanding of the embodiments of the invention. They illustrate embodiments and, in conjunction with the description, help to explain principles and concepts of the invention. Other embodiments and many of the advantages mentioned become apparent in view of the drawings. The elements in the drawings are not necessarily shown to scale.

In the drawings, like, functionally equivalent and identically operating elements, features and components are provided with like reference signs in each case, unless stated otherwise.

### DETAILED DESCRIPTION

One or more example embodiments provides a computer-implemented method for processing clinical data by an artificial intelligence algorithm grid generated by a relevance grid builder and executed for a workflow step of a predefined clinical workflow of a customer to produce an intermediate clinical result having a clinical relevance evaluated by a relevance detector to determine iteratively another artificial intelligence algorithm grid to be executed for the next workflow step of said clinical workflow until configurable end conditions are met.

The clinical relevance of clinical results generated by artificial intelligence (AI) algorithms in a digital health platform refers to the degree to which AI-driven insights, predictions, or recommendations impact patient care, diagnosis, treatment decisions, and overall clinical workflows. In a digital health platform, the clinical relevance of AI-generated results means that these outputs must be accurate, actionable, integrated, compliant, and trustworthy, ultimately enhancing diagnostic confidence, treatment precision, and patient care.

In a possible embodiment the clinical relevance comprises an impact for the clinical workflow and/or comprises an outcome of the clinical workflow and/or comprises a clinical priority and/or comprises a quality or certainty of the intermediate clinical result.

There are different possibilities to evaluate the clinical relevance. In a possible embodiment the determination of the clinical relevance can be performed rule based, e.g. on the basis of medical guidelines. For example a higher BI-RADS Score output by the algorithm can comprise automatically a higher impact or priority. The algorithm can determine the reliability itself, for instance through out-of-distribution detection or by indication of a confidence metric.

Clinical relevance can be modelled also through the diagnostic task of the respective clinical workflow. For example a poly-trauma patient in an emergency department of a hospital has in comparison to a routine scan a completely different prioritisation which consequently requires the application of fast and immediately available algorithms.

The clinical workflow involves in a possible embodiment the processing of medical image data and can concern grid layers of different image processing steps within the clinical workflow which in turn can be executed by different available algorithms. These algorithms can provide image processing intermediate results of the same quality but may comprise different characteristics with respect to deployment, latency, computational costs etc. The layers, i.e. the type of algorithms, can be predetermined by the formulated issue of the clinical workflow. A specific issue can require an execution of analytical steps in conformity with corresponding guidelines. Moreover intermediate results can suggest completely new layers (incidental finding).

Algorithms of different types can be applied. For instance an algorithm is often applied initially to pre-process image data to remove artefacts. Depending on the formulated issue or task of the clinical workflow a segmentation can be performed. Subsequently a detection algorithm to detect a finding is applied. Then other algorithms for performing diagnosis by a user or to generate a diagnostic report can be incorporated. The type of image data can be specified. The method for processing clinical data can be applied in different medical fields, in particular in the field of radiology or in the field of pathology.

The clinical workflow provides a tangible clinical result. For example analysing medical image data provides a diagnostic result.

An intermediate clinical result having a clinical relevance is evaluated by a relevance detector to determine iteratively another artificial intelligence algorithm grid to be executed for the next workflow step of said clinical workflow. Based on the clinical relevance of the intermediate clinical result a selection of an algorithm can be performed from a group of different available algorithms which are suited to perform the next workflow step of the clinical workflow and which comprise different characteristics and requirements (for instance with respect to availability, computation time, accuracy and regulatory requirements etc.).

Processing of clinical data by an artificial intelligence algorithm grid can take into account the technical environment and/or state of a health related system. This includes the availability of algorithms as well as technical characteristics of the available algorithms. The processing can also take into account the clinical relevance modulated by intermediate clinical results.

The artificial intelligence algorithm grid used for the next workflow step is generated dynamically on the fly based on intermediate clinical results and their clinical relevance and based on other clinical data by a relevance grid builder.

The built artificial intelligence algorithm grid associated with a workflow step comprises artificial intelligence algorithms connected to each other in grid, where output data of an artificial intelligence algorithm within the grid can form input data of another artificial intelligence algorithm within the grid.

In a possible embodiment the artificial intelligence algorithm grid may besides its artificial intelligence algorithms also include other programmed processing routines connected within the grid.

The clinical functionality provided by the method according to one or more example embodiments is available over the complete workflow of a customer wherever it is needed.

The method according to one or more example embodiments provides availability of AI processing in all deployment environments. Installation and updates can be done fast, and for AI processing only.

The method according to one or more example embodiments can perform a parallel and/or sequential execution of a chain of artificial intelligence algorithms based on clinical relevance of their clinical results.

AI processing configuration optimizes the customer workflow and enables flexible, case driven execution, giving up inflexible resource requirements and deployments, and yield clinical results based on customer needs. AI results can be constantly improved by supporting an ML Ops approach, which allows to get a feedback about the performance of an executed artificial intelligence algorithm.

The clinical results produced by executed artificial algorithms provide information about clinical relevance (priority, etc.) to optimize the workflow of the physician. The physician can react to those relevant findings with high priority.

The method according to one or more example embodiments provides a pluggable relevance-based artificial intelligence grid which ensures that all necessary AI processing capabilities are available based on the customer workflows and common for all products.

The computer-implemented method makes use of a pipelined relevance grid approach with an automatic detection and an on-the-fly (re-)calculation of a pipelined execution of grids of artificial intelligence algorithms to be executed, governed and optimized by input data and controlled by an on-the-fly evaluation of intermediate clinical results and by data stored in secondary information sources.

In a possible embodiment of the computer-implemented method for processing clinical data according to one or more example embodiments the clinical relevance of an intermediate clinical result produced by an artificial intelligence algorithm grid is evaluated by the relevance detector to either instruct the relevance grid builder to generate another artificial intelligence algorithm grid to be executed for the next workflow step of said clinical workflow or to perform back propagation to a previous workflow step of said clinical workflow. Backpropagation allows for generating more accurate clinical results in an iterative routine.

In a possible embodiment of the computer-implemented method for processing clinical data according to one or more example embodiments the relevance grid builder selects appropriate Artificial Intelligence algorithms for the generated Artificial Intelligence algorithm grid based on data stored in at least one repository and/or based on available input data. This allows for a more focused and efficient processing of clinical data.

In a possible embodiment of the computer-implemented method for processing clinical data according to one or more example embodiments the repository comprises a grid strategy repository containing algorithmic capabilities of Artificial Intelligence algorithms in a machine-readable format. This makes it possible for the relevance grid builder to form the artificial intelligence algorithm grid for the next workflow step using the available most suited and capable artificial intelligence algorithms.

In a possible embodiment of the computer-implemented method for processing clinical data according to one or more example embodiments the detected clinical relevance of the intermediate clinical result produced by an Artificial Intelligence algorithm grid is stored in a relevance grid history repository containing artificial intelligence data of patients and adherent relevance grid configuration data. In this way the clinical relevance stored in the relevance grid history repository is available for the relevance grid builder to shape and optimize the artificial intelligence algorithm grid for the next workflow step taking also into account the stored clinical relevance of the intermediate clinical result provided by the current artificial intelligence algorithm grid executed for the current workflow step.

In a possible embodiment of the computer-implemented method for processing clinical data according to one or more example embodiments the detected clinical relevance of the intermediate clinical result produced by an artificial Intelligence algorithm grid is ranked to provide a relevance ranking processed to perform a worklist prioritization. This allows a user such as a physician to focus on the most relevant clinical results,

In a possible embodiment of the computer-implemented method for processing clinical data according to one or more example embodiments the repository comprises an artificial intelligence imaging repository containing customer configuration data of the customer.

In a possible embodiment of the computer-implemented method for processing clinical data according to one or more example embodiments the repository comprises an auxiliary artificial intelligence repository containing log data created by artificial intelligence algorithms while processing input data.

In a possible embodiment of the computer-implemented method for processing clinical data according to one or more example embodiments the intermediate clinical result produced by an artificial intelligence algorithm grid is stored temporarily and used as input for another artificial intelligence algorithm. This allows for a high degree of automation for data processing and increases the efficiency of processing clinical data during a customer workflow.

In a possible embodiment of the computer-implemented method for processing clinical data according to one or more example embodiments only artificial intelligence algorithms having a regulatory clearance are executed. In this way different regional regulatory rules can be observed.

In a possible embodiment of the computer-implemented method for processing clinical data according to one or more example embodiments the artificial intelligence algorithms are deployed in a cloud, on edge devices or on-premises. This allows an implementation of the computer-implemented method in a wide variety of use cases and health related systems.

In a possible embodiment of the computer-implemented method for processing clinical data according to one or more example embodiments the artificial intelligence algorithms comprises trained deep learning algorithms, in particular convolutional neural networks.

In a possible embodiment of the computer-implemented method for processing clinical data according to one or more example embodiments deployed artificial intelligence algorithms are updated and additional artificial intelligence algorithms are plugged-in or removed during runtime of the computer-implemented method depending on the detected clinical relevance of the intermediate clinical results and/or depending on the clinical workflow. Hot-plugging provides a better and faster adaptability to the workflow and an easier updating of the digital health platform.

In a possible embodiment of the computer-implemented method for processing clinical data according to one or more example embodiments the executed artificial intelligence algorithms provide metering data. This allows measuring the performance of the executed artificial intelligence algorithms and helps in the selection of suitable and efficient artificial intelligence algorithms by the relevance grid builder for building and optimizing the artificial intelligence algorithm grid for the next workflow step.

In a possible embodiment of the computer-implemented method for processing clinical data according to one or more example embodiments the clinical workflow comprises workflow steps including image generations steps for generation of clinical images and/or image processing steps for processing of clinical images and/or image evaluation steps for evaluation of clinical images.

In a possible embodiment of the computer-implemented method for processing clinical data according to one or more example embodiments the artificial intelligence algorithm grid is generated automatically by the relevance grid builder for execution of a workflow step of the predefined clinical workflow by assembling (or selecting) suitable artificial intelligence algorithms which are selected from available artificial intelligence algorithms depending on selection criteria including algorithmic characteristics and/or algorithmic capabilities of the available artificial intelligence algorithms and depending on other restrictions including available processing capabilities, available data storage capacities and allowed running times. The applied selection criteria and/or the applied restrictions can be preconfigured. Assembling may comprise integrating the respective artificial intelligence algorithm into the artificial intelligence algorithm grid.

One or more example embodiments also provides an AI-powered clinical decision support tool adapted to perform the computer-implemented method for processing clinical data according to one or more example embodiments.

One or more example embodiments further provides a computer-program product storing a program adapted to perform the computer-implemented method for processing clinical data according to one or more example embodiments when being executed on a processor.

One or more example embodiments further provides a digital health platform comprising an artificial intelligence engine adapted to perform the computer-implemented method according to one or more example embodiments.

The AI-powered clinical decision support software tools of the digital health platform are designed to assist radiologists in image interpretation, segmentation, quantification, and anomaly detection across multiple imaging modalities, including CT, MRI, and X-ray. The digital health platform leverages deep learning algorithms, particularly convolutional neural networks (CNNs), to automate and enhance various aspects of medical imaging analysis. The digital health platform is cloud-based and integrates seamlessly with Picture Archiving and Communication Systems (PACS) and Radiology Information Systems (RIS) via DICOM (Digital Imaging and Communications in Medicine) standards.

Where appropriate, the above-mentioned configurations and developments can be combined implementations can be combined with each other as desired, as far as this is reasonable. Further possible configurations, developments and implementations of the invention also include combinations, which are not explicitly mentioned, of features of example embodiment which have been described previously or are described in the following with reference to the embodiments. In particular, in this case, a person skilled in the art may also add individual aspects as improvements or supplements.

Advantageous configurations and developments emerge from the further dependent claims and from the description with reference to the figures of the drawings.

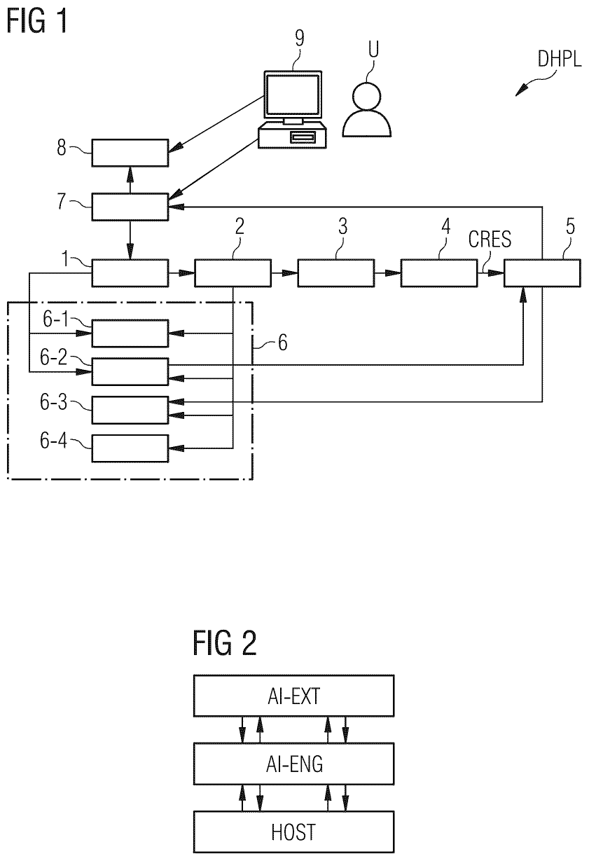

One or more example embodiment further provides a digital health platform DHPL comprising an artificial intelligence engine AI-ENG adapted to perform the computer-implemented method according to one or more example embodiments. FIG. 1 shows a possible exemplary embodiment of a digital health platform DHPL including components adapted to perform the computer-implemented method according to one or more example embodiments. FIG. 1 illustrates schematically the approach to dynamically integrate relevance-based results into the artificial processing in a way that a relevance ranking can also be shown to a physician or other user U via a workstation or via another user interface for improving a work routine of the physician or user U. A fully automated processing does in a possible embodiment not require any interaction with a physician and sends clinical results CRES directly to the required next system component. For reading by a physician, it is required that all data is shown on his workstation. FIG. 1 shows the technical infrastructure for executing artificial intelligence processing as an integral part of a smart imaging value chain which represents the clinical workflow WF of a physician and enables a seamless integration.

As can be seen in the block diagram of FIG. 1 the illustrated digital health platform DHPL comprises a grid input analyzer 1 adapted to receive DICOM data from a Data Input/Output Unit 7. The analyzed data is supplied to a relevance grid builder 2 having access to a data base including several repositories 6-1, 6-2, 6-3, 6-4 as shown in FIG. 1. Clinical data is processed by artificial intelligence algorithms AI-ALs of an Artificial Intelligence algorithm grid AI-ALG built by the relevance grid builder 2. The artificial intelligence algorithm grid AI-ALG is run on a grid executor 3 to trigger the execution of its artificial intelligence algorithms AI-ALs on processing means 4 (e.g., a processor) to produce an intermediate clinical result CRES.

The produced intermediate clinical result produced by the currently executed artificial intelligence algorithm grid AI-ALG for a workflow step WF-STi comprises a clinical relevance CREL evaluated by a relevance detector 5 to determine iteratively another Artificial Intelligence algorithm grid AI-ALG i+1 to be executed for the next workflow step WF-STi+1 of said clinical workflow WF until configurable end conditions are met.

According to one or more example embodiments a computer-implemented method for processing clinical data by an artificial intelligence algorithm grid AI-ALG generated by a relevance grid builder 2 and executed for a workflow step of a predefined clinical workflow of a customer to produce an intermediate clinical result CRES having a clinical relevance CREL. The clinical relevance CREL is evaluated by a relevance detector 5 to determine iteratively another Artificial Intelligence algorithm grid to be executed for the next workflow step of said clinical workflow until configurable end conditions are met. In a possible embodiment of the computer-implemented method for processing clinical data the detected clinical relevance CREL of the intermediate clinical result CRES produced by an artificial intelligence algorithm grid AI-ALG is ranked to provide a relevance ranking processed to perform a worklist prioritization. A clinical relevance ranking can be calculated by a ranking algorithm using metrics, in particular metering data provided by executed artificial intelligence algorithms, and other clinical data stored in a repository. The clinical results CRES are supplied by the detector 5 along with its relevance ranking to the DICOM Input/output Unit 7 of the digital health platform DHPL as illustrated in FIG. 1. Data con be converted by a block 8 into FHIR Data. The DICOM Input/output Unit 8 is connected via a data network to the workstation 9 of a user such as a physician or radiologist. The user can read the received clinical results along with their relevance ranks on a screen of the workstation 9.

The method according to one or more example embodiments implements a pipelined relevance grid approach and enhances the existing approach by introducing the following core ideas:

The method according to one or more example embodiments introduces using a hot-pluggable relevance grid, which allows to add and remove, schedule, and reschedule, AI processing dynamically based on the data in the customer workflows and the clinical relevance CRES of the produced AI results.

The method according to one or more example embodiments introduces cover the runtime challenges such as providing the AI processing where it is needed, i.e. in a cloud, on an edge device or on-premises. The method according to one or more example embodiments can make use of downloadable Artificial Intelligence algorithms. Each Artificial Intelligence processing can be configured so that it fits to the given deployment, based on parameters for GPU/CPU, RAM, and disk usage. This allows setting up the system according to one or more example embodiments for optimizing the given workload in terms of resources and costs.

With the method according to one or more example embodiments an order and type of the AI processing execution can be dynamically determined based on the input data and previous medical clinical AI results and their clinical relevance. To optimize artificial intelligence results, prior intermediate clinical results CRES can be used as additional input for indication of trends over time.

AI clinical results CRES with high criticality and medical relevance can trigger a systemic reaction for prioritized reading by the radiologist e.g. performing a worklist prioritization in the radiology information system. The decision which artificial intelligence algorithm AI-AL will be executed next is made based on current intermediate clinical results CRES. Thereby, the method according to one or more example embodiments introduces an automatic calculated grid AI-ALG of artificial intelligence algorithms AI-ALs for each workflow step of the clinical workflow WF with an optimized runtime structure and a pipeline of such Artificial Intelligence algorithm grids AI-ALGs that is as long and runs as long and iteratively, including back propagation, until configurable end-conditions are met. This approach enables a flexible execution network for artificial intelligence processing.

In a preferred embodiment only artificial intelligence algorithms AI-ALs with medical clearance (approval by authorities) shall be executed. Each AI algorithm forms a medical entity which is released for specific countries / regions. It is the responsibility of the Artificial Intelligence Engine to trigger only cleared medical devices/algorithms. For example in a cloud deployment all relevant artificial intelligence processing entities can be deployed. However, only those versions of the artificial intelligence processing components having been regulatory cleared in the respective country are executed.

As also illustrated in FIG. 4 in a possible embodiment of the computer-implemented method for processing clinical data the clinical relevance CREL of an intermediate clinical result CRES produced by a currently executed artificial intelligence algorithm grid AI-ALGi is evaluated by the relevance detector 5 to either instruct the relevance grid builder 2 to generate another Artificial Intelligence algorithm grid AI-ALG i+1 to be executed for the next workflow step WFi+1 of said clinical workflow WF or to perform back propagation BPROP to a previous workflow step of said clinical workflow WF.

In a possible embodiment of the computer-implemented method for processing clinical data the relevance grid builder 2 selects appropriate Artificial Intelligence algorithms AI-ALs for the generated Artificial Intelligence algorithm grid AI-ALG based on data stored in at least one repository of the data base 6 and/or based on available input data.

In a possible embodiment of the computer-implemented method for processing clinical data the repository 6 comprises an artificial intelligence imaging repository 6-1 containing customer configuration data of the customer. The AI Imaging repository 6-1 can contains the configuration for data from different scanners from different vendors as they have different capabilities and therefore provide different input data. Furthermore, each customer has its own data model for providing the data in DICOM. (e.g. the DICOM tag series description can contain relevant data for routing the input data to the appropriate AI processing). This variability can be configured to meet the requirements of the customer environment and are an enabler for a seamless integration into the customer's setup. A guided integration into the multitude of products and the multi-product deployments on-site is achieved, such that meta-data and image data on the input side reaches semantic compatibility with the relevance grid, such that the dynamic grid execution path calculation inside the AI engine has more input, and such that all data at the output side reaches compatibility with on-site input requirements for data integration into the multitude of products (e.g. in terms of DICOM and FHIR medical domain standards).

The repository 6-1 can also contain in a possible embodiment information about licensing and regulatory approval. Each AI processing component which typically is a medical component requires a regulatory approval for a given country or region. The AI Engine ensures that only medical components or processing resources are used for AI processing that have a regulatory approval for the customer location. Therefore, the AI Engine can be connected to a cloud-based subscription management unit in a digital backbone of the digital health platform DHPL which provides all regulatory information as part of an entitlement. A customer related license information can also form part of the entitlement for an AI processing component, which the AI Engine then evaluates. FIHR (Fast Healthcare Interoperability Resources) is a standard for exchanging healthcare information electronically, designed to make it easier to share and integrate health-related data from different data sources. FHIR data essentially refers to health data formatted according to this standard, which aims to improve interoperability, flexibility, and efficiency in healthcare information exchange. For example in the cloud all available AI processing is deployed but only those to which the customer has an entitlement are executed. The AI engine can pick in a possible embodiment the correct version for each country or region the customer is in.

In a possible embodiment of the computer-implemented method for processing clinical data the repository comprises a grid strategy repository 6-2 containing algorithmic capabilities of Artificial Intelligence algorithms AI-ALs in a machine-readable format. The grid strategy repository 6-2 contains algorithmic capabilities in a machine-readable format of the underlying algorithm of the AI processing component. This allows to generate the grids and supervise AI algorithm execution automatically. Each algorithm can be trained for a given set of supported DICOM capabilities of the input data for creating the best clinical results CRES. Those capabilities are evaluated by the Artificial Intelligence Engine AI-ENG together with the provided input data (e.g. modality, scan parameters like slice thickness, transfer syntax and more).

In a possible embodiment of the computer-implemented method for processing clinical data the detected clinical relevance CREL of the intermediate clinical result CRES produced by an Artificial Intelligence algorithm grid AI-ALG is stored in a relevance grid history repository 6-3 containing artificial intelligence data of patients and adherent relevance grid configuration data. The relevance grid history repository 6-3 stores historical AI data of a patient and adherent relevance grid configuration, such that it can be used for future processing. This enables the system to compare multiple time series of data and the evolution of it. This can also impact the calculated relevance rank of the actual processing.

In a possible embodiment of the computer-implemented method for processing clinical data the repository 6 comprises an auxiliary artificial intelligence repository 6-4 containing log data created by artificial intelligence algorithms while processing input data. The auxiliary AI repository 6-4 is provided to support monitoring the system from an organizational aspect as well as from a ML Ops aspect. AI processing creates anonymized log data to improve the quality of the algorithms and the quality of the AI results. The logs are gathered in a centralized data sink for further analysis. The log data can be transferred to a central data lake in the cloud for further analysis by data scientists and developers.

The relevance grid builder 2 is responsible for creating the relevance grid, for each step, and for selecting the appropriate AI algorithms AI-ALs. The necessary information is read from the various data sources shown in FIG. 1. As the data in the Grid Strategy Repository 6-2 and Relevance Grid History Repository 6-3 is potentially updated for each processing the relevance grid is changing and improving constantly based on feedback from the AI processing.

In the pipelined relevance grid clinical results CRES are created based on the capabilities of an AI processing/algorithm, customer configuration, input data and regulatory clearance (see FIG. 1 and FIG. 4). Clinical results CRES of an algorithm can be the input for other artificial processing, e.g. based on a rule set. This allows the system to dynamically create a grid of available AI processing to create outcome with high clinical relevance CREL. The decision which artificial intelligence algorithm AI-AL must be executed next is made based on current clinical results. Thereby, the method introduces an automatic calculated grid of AI algorithms for each step with optimized run-time structure and a pipeline of such grids that is as long and runs as long und iteratively, including back propagation, until configurable end-conditions are met.

The relevance detector 5 is adapted to analyze the AI clinical results and processes the relevance of the AI clinical results in a way that the clinical results CRES are tagged with the appropriate clinical relevance CREL. This enables the system to calculate the next focused grid step or not, and finally to provide more information to an external system. E.g. in a RIS the data can be shown with higher priority in the RIS worklist. It also enables the radiologist to focus on the important clinical findings first as they can be visualized in the user interface. The detected clinical relevance CREL is stored in the relevance grid history repository 6-3 so that it can dynamically serve as an input for the relevance grid builder 2 for upcoming processing.

The intermediate clinical result CRES produced by an artificial intelligence algorithm grid AI-ALG can be stored temporarily in an intermediate data memory and can be used as input for another Artificial Intelligence algorithm AI-ALG.

In a possible embodiment of the computer-implemented method for processing clinical data only artificial intelligence algorithms having a regulatory clearance are executed. The clearance can be performed during deployment before execution or during runtime on the basis of predefined rules.

In a possible embodiment of the computer-implemented method for processing clinical data the artificial intelligence algorithms AI-AL comprises a trained deep learning algorithm, in particular a convolutional neural network (CNN).

The deployed artificial intelligence algorithms AI-ALs are updated and additional artificial intelligence algorithms AI-ALs can be plugged-in or removed during runtime of the computer-implemented method depending on the detected clinical relevance CREL of the intermediate clinical results CRES and/or depending on the clinical workflow. The system can plug-in new AI processing or update existing processing during runtime so that a customer can benefit immediately from the latest innovation (Dev Ops approach).

In a possible embodiment of the computer-implemented method for processing clinical data the executed artificial intelligence algorithms AI-ALs provide metering data. The infrastructure in the Artificial Intelligence Engine AI-ENG provides in a preferred embodiment metering data for all artificial intelligence processing routines so that a 100% flexible execution model can be technically supported, e.g. for the key aspects'resources, deployment types, data-to-product integration, and on-demand AI-algorithm selection. This can be vital to run the right AI-Algorithm AI-AL on demand, and such that a pay-per-use business model can be implemented. That approach enables the customer (especially in the cloud) to pay only for services he has actively used.

An agnostic deployment of AI processing routines provides the same clinical AI results on all systems in a hospital, so that it is not relevant for the physician how and where the clinical capabilities are provided. The artificial intelligence algorithms AI-ALs are deployed in a cloud, on an edge devices or on-premises. The same AI processing can be executed in all deployments and adapted to their environment. For example RAM, CPU, GPU are different on a scanner than in a cloud deployment but produce the same clinical results CRES with the same clinical relevance CREL. To support an agnostic deployment, each deployment scenario is supported in a transparent and portable way by the Artificial Intelligence Engine AI-ENG, so that the AI processing can be integrated seamlessly into the customer's work routine.

Using a pipelined relevance grid clinical results CRES are created based on the capabilities of an AI processing/algorithm, customer configuration, input data and regulatory clearance (see FIG. 1). Clinical results CRES of an algorithm can be the input for other AI processing, e.g. based on a rule set. This allows the system to dynamically create a grid of available artificial intelligence processing algorithms to create outcome with high clinical relevance CREL. The decision which artificial intelligence algorithm AI-AL will be executed next is made based on current clinical results CRES produced by the current artificial intelligence algorithm grid AI-ALG. Thereby, the method introduces an automatic calculated grid of AI algorithms for each step with optimized run-time structure and a pipeline of such grids that is as long and runs as long und iteratively, including back propagation, until configurable end-conditions are met.

In a possible embodiment of the computer-implemented method for processing clinical data the clinical workflow WF comprises workflow steps including image generation steps for generation of clinical images and/or image processing steps for processing of clinical images and/or image evaluation steps for evaluation of clinical images.

In a possible embodiment of the computer-implemented method for processing clinical data the artificial intelligence algorithm grid AI-ALG is generated automatically by the relevance grid builder 2 for execution of a workflow step of the predefined clinical workflow WF by assembling suitable artificial intelligence algorithms ALs (which are selected from available artificial intelligence algorithms AI-Als) into the grid AI-ALG. The selection can be performed depending on selection criteria and/or depending on restrictions. The applied selection criteria can for instance include algorithmic characteristics and/or algorithmic capabilities of the available artificial intelligence algorithms AI-ALs. The restrictions can include available processing capabilities, available data storage capacities and allowed running times. The applied selection criteria and/or the applied restrictions can be preconfigured. The selection and assembly can be performed fully-automatically or semi-automatically with the intervention of a user.

The selection and assembly of the suitable artificial intelligence algorithms AI-ALs by the grid builder 2 to form an artificial intelligence algorithm grid AI-ALG for the next workflow step WF-ST i+1 of the currently executed clinical workflow WF can be performed in a possible embodiment on the fly in real time taking also into account the clinical relevance CREL of the clinical results CRES provided by the artificial intelligence algorithm grid AI-ALG of the momentary workflow step WF-ST i. Assembling of the selected artificial intelligence algorithms AI-ALs into the grid AI-ALG can be performed by the grid builder 2 in a possible embodiment by connecting interfaces of the selected artificial intelligence algorithms AI-ALs with each other.

The performance of an assembled and executed artificial intelligence algorithm grid AI-ALG can in a possible embodiment be measured and/or evaluated. An Artificial intelligence algorithm grid AI-ALG with high performance can be stored in a repository for preassembled Artificial intelligence algorithm grids AI-ALGs and can be loaded on demand by the grid builder 2 for modification of the loaded Artificial intelligence algorithm grid AI-ALG depending on the requirements of the next workflow step of the clinical workflow WF. The modification of the loaded Artificial intelligence algorithm grid AI-ALG can for instance comprise the substitution of an artificial intelligence algorithm AI-AL assembled in the loaded Artificial intelligence algorithm grid AI-ALG by another more suitable available artificial intelligence algorithm AI-AL. The modification may also comprise adding of at least one further artificial intelligence algorithm AI-AL to the loaded Artificial intelligence algorithm grid AI-ALG or the removal of an artificial intelligence algorithm AI-AL from the loaded Artificial intelligence algorithm grid AI-ALG.

Based on the clinical relevance CREL of the AI results further clinically relevant use cases can be triggered like for example the prioritization of a worklist (also in external systems like a RIS) so that a physician gets a visual indication about the clinical relevance. Furthermore, an ML Ops approach can be supported in a way that a feedback loop about the processed data and the created clinical results CRES is established which enables the digital health platform to improve the quality of AI processing based on real-live data.

Each artificial intelligence processing algorithm can be considered as a separate medical entity. The Artificial Intelligence Engine AI-ENG provides all necessary interfaces for supporting independent and portable integration and execution of medical devices. This setup also enables different deployments of different medical devices based on country regulations.

The method according to one or more example embodiments has the advantage that it creates a single source of truth for AI processing for all technical and clinical aspects that are relevant to AI processing in largest clinical environments. Moreover the method has the additional advantage the Radiology-as-a-Service with distributed sites on multiple continents can be fully supported, because the mutual alignment of artificial intelligence algorithms AI-ALs across different sites is enabled. According to one or more example embodiments requirements from a technical to use smarter, more focused, faster AI-algorithms AI-ALs with smaller resource footprints are resolved with a smarter and self-adaptive grid solution.

Artificial Intelligence encompasses a universe of techniques and technologies. According to one or more example embodiments it can be ensured that all types of artificial intelligence can be integrated, local and remote, due to the agnostic deployment capability, due to the grid strategies, due to the pre-calculation of one step with a relevance grid at a time, which then is repeated with different grids until completion.

FIG. 2 shows the technical context of AI processing for making it available in all required deployments including deployment in a cloud, on edge devices, on-premises and on scanners. All capabilities and inventions described above are provided by the AI Engine component and the AI automation components (AI-EXT) which perform the AI processing. The artificial intelligence engine AI-ENG provides a processing framework forming an integration layer between clinical applications and hosts. The integration layer facilitates clinical application software to run on any host.

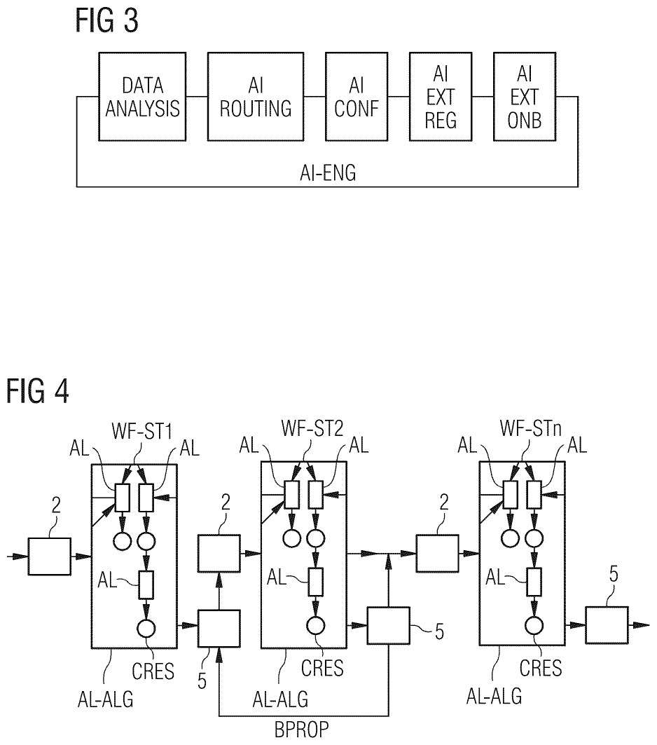

FIG. 3 shows an integration layer of the AI Engine AI-ENG which provides all capabilities to deploy, configure and to run artificial intelligence processing algorithms on all hosts. The AI engine AI-ENG provides a Data Role Service DRS, AI routing and coordination, AI Configuration (Engine and AI Extensions) and an AI Onboarding and Extension Registry. The Onboarding and Extensions Registry AI-EXT-ONB as illustrated in FIG. 3 provides capabilities for a dynamic onboarding of AI processing routines (container including algorithms and metadata) and all necessary information for the relevance detector 5 to create AI calculated clinical results with an associated calculated relevance rank. FIG. 3 shows all the required components of the Artificial Intelligence Engine AI-ENG which are required to fulfill different use cases. The artificial intelligence engine AI-ENG comprises an integration layer for all artificial intelligence processing routines to guarantee the same calculated clinical results in all required deployments. Depending on the use cases these core components are deployed in all customer use cases.

In FIG. 4 illustrates schematically how for each workflow step WF-STi the relevance grid builder 2 shapes the optimized AI-algorithm grid AI-ALG for the next workflow step, WF-STi+1.Then the relevance detector 5 first matches the AI results to the current progress made in the use case, and then takes the decision to instruct the relevance grid builder 2 for the next workflow step WF-STi+1 or decide for performing a backpropagation BPROP, or to decide for a termination or an emergency abortion. The method according to one or more example embodiments can automatically re-calculate and repeat steps, and also back-propagate all types of results to former steps (e.g. to support smart chaining of AI-algorithms AI-ALs). For example in a Chest CT with a number of AI-algorithms, each of which is needed or not, depending on the case (cough or cancer, for example) the intermediate AI results can cause that e.g. all other AI-algorithms are skipped if the AI-algorithms for the lung tip have detected a lung nodule of category 4X.

Although the present invention has been described in the above by way of embodiments, it is not limited thereto, but rather can be modified in a wide range of ways. In particular, the invention can be changed or modified in various ways without deviating from the core of the invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for processing clinical data by an artificial intelligence algorithm grid generated by a relevance grid builder, the method comprising:
executing the artificial intelligence algorithm grid for a workflow step of a predefined clinical workflow of a customer to produce an intermediate clinical result having a clinical relevance evaluated by a relevance detector; and
determining iteratively another artificial intelligence algorithm grid to be executed for a next workflow step of the clinical workflow until configurable end conditions are met.

2. The method of claim 1, wherein the evaluating by the relevance detector includes,
instructing the relevance grid builder to generate the another artificial intelligence algorithm grid to be executed for the next workflow step of the clinical workflow, or performing back propagation to a previous workflow step of the clinical workflow.

3. The method of claim 1, wherein the relevance grid builder selects appropriate artificial intelligence algorithms for the generated artificial intelligence algorithm grid based on data stored in at least one repository.

4. The method of claim 3, wherein the repository comprises at least one of:
an artificial intelligence imaging repository containing customer configuration data of the customer;
a grid strategy repository containing algorithmic capabilities of artificial intelligence algorithms in a machine-readable format; or
an auxiliary artificial intelligence repository containing log data created by artificial intelligence algorithms while processing input data.

5. The method of claim 1, wherein the clinical relevance of the intermediate clinical result is stored in a relevance grid history repository containing artificial intelligence data of patients and adherent relevance grid configuration data.

6. The method of claim 1, wherein the clinical relevance of the intermediate clinical result is ranked to provide a relevance ranking to perform a worklist prioritization.

7. The method of claim 1, wherein the intermediate clinical result is stored temporarily and used as input for another artificial intelligence algorithm.

8. The method of claim 1, wherein only artificial intelligence algorithms having a regulatory clearance are executed.

9. The method of claim 1, wherein artificial intelligence algorithms for the artificial intelligence algorithm grid are deployed in a cloud, on edge devices or on-premises.

10. The method of claim 9, wherein the artificial intelligence algorithms comprise trained deep learning algorithms.

11. The method of claim 1, wherein deployed artificial intelligence algorithms are updated and additional artificial intelligence algorithms are plugged-in or removed during runtime of the computer-implemented method based on at least one of the clinical relevance of the intermediate clinical results or the clinical workflow.

12. The method of claim 1, wherein executed artificial intelligence algorithms for the artificial intelligence algorithm grid provide metering data.

13. The method of claim 1, wherein the clinical workflow comprises workflow steps including at least one of image generations steps for generation of clinical images, image processing steps for processing of clinical images, or image evaluation steps for evaluation of clinical images.

14. The method of claim 1, wherein the artificial intelligence algorithm grid is generated automatically by the relevance grid builder for execution of the workflow step of the predefined clinical workflow by assembling suitable artificial intelligence algorithms which are selected from available artificial intelligence algorithms based on selection criteria including at least one of algorithmic characteristics or algorithmic capabilities of the available artificial intelligence algorithms and further based on other characteristics including available processing capabilities, available data storage capacities and allowed running times.

15. A digital health platform comprising:
an artificial intelligence engine configured to perform the method of claim 1.

16. An artificial intelligence (AI)-powered clinical decision support tool configured to perform the method of claim 1.

17. A non-transitory computer readable medium storing instructions that, when executed by a processor, cause the processor to perform the method of claim 1.

18. The method of claim 10, wherein the artificial intelligence algorithms comprise convolutional neural networks.

19. The method of claim 2, wherein the relevance grid builder selects appropriate artificial intelligence algorithms for the generated artificial intelligence algorithm grid based on data stored in at least one repository.

20. The method of claim 2, wherein deployed artificial intelligence algorithms are updated and additional artificial intelligence algorithms are plugged-in or removed during runtime of the computer-implemented method based on at least one of the clinical relevance of the intermediate clinical results or the clinical workflow.
